(19) United States
(12) Patent Application Publication
(10) Pub. No.: US 2026/0232798 A1
(43) Pub. Date: Aug. 13, 2026

(54) PLASMACYTOID DENDRITIC CELLS AND IMMUNE CHECKPOINT INHIBITOR FOR CANCER TREATMENT

(71) Applicant: PDC LINE PHARMA, Liége (BE)

(72) Inventors: Joël PLUMAS, Grenoble (FR); Laurent LEVY, Albigny-sur-Saône (FR)

(73) Assignee: PDC LINE PHARMA, Liége (BE)

(21) Appl. No.: 19/156,462

(22) PCT Filed: Feb. 17, 2023
(86) PCT No.: PCT/EP2023/054091

§ 371 Date: Aug. 14, 2025

(51) Int. Cl. A61K 40/19; A61K 39/00; A61K 39/395; A61K 40/24; A61K 40/42; A61K 45/06; A61P 35/00

(52) U.S. Cl. CPC A61K 40/19; A61K 39/3955; A61K 40/24; A61K 40/424; A61K 40/4268; A61K 40/4269; A61K 45/06; A61P 35/00; A61K 2039/505

(57) ABSTRACT

A method for treating and/or preventing cancer in a subject. The method includes administering to the subject a population of plasmacytoid dendritic cells and an immune checkpoint inhibitor. The population of plasmacytoid dendritic cells and the immune checkpoint inhibitor may be administered separately or in combination, and in any order.

### FIELD OF INVENTION

The present invention relates to methods for treating and/or preventing cancer using a combination of plasmacytoid dendritic cells and immune checkpoint inhibitor.

### BACKGROUND OF INVENTION

Cancer vaccine approach stimulating the immune system is one of the main strategies worldwide to fight against cancer. The main cancer vaccine approach consists of priming and activating the patient's tumor-specific cytotoxic CD8+ T lymphocytes with dendritic cells (DCs) since they are professional antigen-presenting cells. However, cancer vaccines based on autologous DCs have proven difficult to produce and have shown little clinical benefit. Allogeneic DC-based vaccines are proposed as alternatives to their autologous counterparts because they overcome several issues, including the lack of reproducibility and the difficulty of production from patients' blood. In addition, the allogeneic response due to the expression of mismatched HLA on allogeneic DCs is expected to generate an activation stimulus that strengthens the stimulation of antigen-specific CD8+ T cells (ASTCs).

In the context of melanoma, the inventors previously showed that plasmacytoid dendritic cells (PDC) loaded with peptides derived from four melanoma-associated antigens activated the rare tumor ASTCs present in the blood and, more importantly, in the tumors of patients. These ASTCs proliferated, switched from a naïve to a memory phenotype, displayed cytotoxic potential, and killed autologous melanoma cells. Vaccination experiments in humanized mice also demonstrated the immunostimulatory and tumoricidal potential of PDC-based vaccines (Aspord C. et al, PLoS ONE, 2010). Moreover, the inventors reported a first-in-human phase I/II study with melanoma patients (clinical trial number NCT01863108) treated with PDC*line cells loaded with four melanoma antigen-derived peptides (Charles J. et al, OncoImmunology 2020). The product, named “Genius Vac-Mel4”, was safe and well tolerated with encouraging signs of clinical activity. Strikingly, a significant increase in the frequency of circulating memory ASTCs was observed, thereby demonstrating the priming and expansion of ASTCs by the Genius Vac-Mel4 vaccine in humans.

Another strategy for cancer treatment consists of immunotherapies with immune checkpoint inhibitors (ICIs), such as antibodies against the programmed cell death protein-1 (anti-PD-1) or its ligand PD-L1. This strategy has proven notably efficient in the case in lung cancer, which is the leading cause of cancer-related mortality worldwide, with an estimated 1.8 million cancer deaths in 2020. Unresectable metastatic non-small-cell lung cancer (NSCLC) patients without genetic alterations (which represents 80-85% of lung cancer cases) are now treated, in first-line treatment, with ICIs in monotherapy or in combination with chemotherapy depending on the level of tumor PD-L1 expression.

However, both strategies appear not to be optimal. First, despite the attempts of the scientific community to improve DC-based cancer vaccines, there is still a need to improve this strategy in order to potentiate the ASTCs immune response in patient in need. Second, the observed clinical benefits with the ICIs remains unsatisfying, as many patients still do not respond or develop resistance to the therapy.

There is thus an unmet medical need to improve these anticancer therapies.

### SUMMARY

This invention relates to method for treating and/or preventing a cancer in a subject in need thereof, comprising administering to the subject the combination of a therapeutically effective amount of a population of plasmacytoid dendritic cells (PDC) and a therapeutically effective amount of an immune checkpoint inhibitor, wherein said population and said inhibitor may be administered separately or in combination, and in any order.

In some embodiments, the immune checkpoint inhibitor is an inhibitor of programmed cell death protein-1 (PD-1) or an inhibitor of the ligand of PD-1 (PDL-1).

In some embodiments, the inhibitor of PD-1 is an anti-PD-1 antibody or a fragment thereof. In some embodiments, the inhibitor of PD-1 is selected from the group comprising or consisting of pembrolizumab, nivolumab, lambrolizumab, dostarlimab and cemiplimab.

In some embodiments, the inhibitor of PDL-1 is an anti-PDL-1 antibody or a fragment thereof. In some embodiments, the inhibitor of PDL-1 is selected from the group comprising or consisting of atezolizumab, durvalumab and avelumab.

In some embodiments, the cancer is selected from the group comprising or consisting of lung cancer, melanoma, breast cancer, prostate cancer, colon cancer, squamous cell cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulvar cancer, thyroid cancer, hepatic carcinoma, head and neck cancer, and the like.

In some embodiments, the cancer is lung cancer or melanoma. In some embodiments, the cancer is small-cell lung cancer (SCLC) or non-small cell lung cancer (NSCLC). In some embodiments, the cancer is NSCLC.

In some embodiments, the population of PDC is contacted with at least one cancer antigen and/or fragment and/or variant thereof.

In some embodiments, the at least one cancer antigen and/or fragment and/or variant thereof is selected from the group comprising MAGE-A1, MAGE-A2, MAGE-A3, MAGE-A4, MAGE-A9, MAGE-A10, CAMEL (CTAG2), GLULD1 (LGSN), HER2 (ERBB2), MUC1, SURVIVIN (BIRC5), NY-ESO-1, MULTI-MAGE, NY-BR-1, Cyclin D1 (CCND1), PD-L1, CEA, EPCAM, IDO, LY-6K, MUC5AC, and 5T4 (TPBG), preferably the at least one cancer antigen and/or fragment and/or variant thereof is selected from the group comprising MAGE-A1, MAGE-A3, MAGE-A4, MAGE-A9, MUC1, SURVIVIN (BIRC5), NY-ESO-1, CAMEL, MULTI-MAGE, 5T4 (TPBG) and CCND1.

In some embodiments, the population of PDC is administered at a dose from 50,000 cells/antigen and/or fragment and/or variant thereof to 5 million cells/antigen and/or fragment and/or variant thereof, preferably at a dose from 500,000 cells/antigen and/or fragment and/or variant thereof to 5 million cells/antigen and/or fragment and/or variant thereof, more preferably at a dose of about 2 million cells/antigen and/or fragment and/or variant thereof.

In some embodiments, the population of PDC is administered at least one time, preferably at least 3 times, more preferably at least 6 times. In some embodiments, the population of PDC is administered from 1 time to 10 times.

In some embodiments, the immune checkpoint inhibitor is administered at a dose of from 3 to 10 mg/kg.

In some embodiments, the immune checkpoint inhibitor is administered at least one time, preferably at least 3 times, more preferably at least 6 times.

In some embodiments, the immune checkpoint inhibitor and/or said population of PDC are administered intravenously, subcutaneously, intraarterially, or intramuscularly, preferably intravenously or subcutaneously.

In some embodiments, the method further comprises administering to the subject at least one additional anti-cancer agent.

In some embodiments, the method further comprises administering to the subject at least one adjuvant.

In some embodiments, the method further comprises a step of assessing the immune background of the subject by blood sampling.

In some embodiments, the method further comprises a step of assessing the immunogenicity of the subject by blood sampling at least one-time post-vaccination.

The present invention further relates to a combination of a population of plasmacytoid dendritic cells (PDC), and at least one immune checkpoint inhibitor, for use for the treatment and/or prevention of cancer in a subject in need thereof.

The present invention further relates to a population of plasmacytoid dendritic cells (PDC) for use for treating and/or preventing cancer, wherein the population of PDC is for administration or is administered in combination with at least one immune checkpoint inhibitor.

The present invention further relates to a pharmaceutical composition comprising a population of plasmacytoid dendritic cells (PDC) and at least one immune checkpoint inhibitor for treating and/or preventing cancer in a subject in need thereof.

The present invention further relates to a kit comprising a population of plasmacytoid dendritic cells (PDC) and at least one immune checkpoint inhibitor for treating and/or preventing cancer in a subject in need thereof.

### Definitions

In the present invention, the following terms have the following meanings:

“About”, when preceding a figure, means plus or less 10% of the value of said figure.

“And/Or” refers to and encompasses any and all possible combinations of one or more of the associated listed items, as well as the lack of combinations when interpreted in the alternative (“or”).

“Antibody” and “immunoglobulin” may be used interchangeably and refer to a protein having a combination of two heavy and two light chains whether or not it possesses any relevant specific immunoreactivity. “Antibodies” refers to such assemblies which have significant known specific immunoreactive activity to an antigen of interest (e.g., PD-1). The term “anti-PD-1 antibodies” is used herein to refer to antibodies which exhibit immunological specificity for PD-1. Specificity for human PD-1 does not exclude cross-reaction with other species. Antibodies and immunoglobulins comprise light and heavy chains, with or without an interchain covalent linkage between them. Basic immunoglobulin structures in vertebrate systems are relatively well understood. The generic term “immunoglobulin” comprises five distinct classes of antibody that can be distinguished biochemically. Although the following discussion will generally be directed to the IgG class of immunoglobulin molecules, all five classes of antibodies are within the scope of the present invention. With regard to IgG, immunoglobulins comprise two identical light polypeptide chains of molecular weight of about 23 kDa, and two identical heavy chains of molecular weight of about 53-70 kDa. The four chains are joined by disulfide bonds in a “Y” configuration wherein the light chains bracket the heavy chains starting at the mouth of the “Y” and continuing through the variable region. The light chains of an antibody are classified as either kappa (κ) or lambda (λ). Each heavy chain class may be bonded with either a κ or λ light chain. In general, the light and heavy chains are covalently bonded to each other, and the “tail” regions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain. Those skilled in the art will appreciate that heavy chains are classified as gamma (γ), mu (μ), alpha (α), delta (Δ) or epsilon (ε) with some subclasses among them (e.g., γ1-γ4). It is the nature of this chain that determines the “class” of the antibody as IgG, IgM, IgA IgD or IgE, respectively. The immunoglobulin subclasses or “isotypes” (e.g., IgG1, IgG2, IgG3, IgG4, IgA1, etc.) are well characterized and are known to confer functional specialization. Modified versions of each of these classes and isotypes are readily discernable to the skilled artisan in view of the instant disclosure and, accordingly, are within the scope of the present invention. As indicated above, the variable region of an antibody allows the antibody to selectively recognize and specifically bind epitopes on antigens. That is, the light chain variable domain (VL domain) and heavy chain variable domain (VH domain) of an antibody combine to form the variable region that defines a three-dimensional antigen binding site. This quaternary antibody structure forms the antigen binding site presents at the end of each arm of the “Y”. The term “antibody” further extends to or antibody mimetics.

The term “antigen-binding fragment”, as used herein, refers to a part or region of the antibody according to the present invention, which comprises fewer amino acid residues than the whole antibody. An “antigen-binding fragment” binds antigen and/or competes with the whole antibody from which it was derived for antigen binding (e.g., specific binding to PD-1). Antibody antigen-binding fragments encompasses, without any limitation, single chain antibodies, Fv, Fab, Fab′, Fab′-SH, F(ab)′2, Fd, defucosylated antibodies, diabodies, triabodies and tetrabodies.

“At least one” includes 1, 2, 3, 4, 5, 6, 7, 8, 9, 10, 25, 50, 75, 100, 250, 500, 750, 10³, 10⁴, 10⁵, 10⁶, 10⁷, 10⁸, 10⁹, 10¹⁰, 10¹¹, 10¹², 10¹³, 10¹⁴, 10¹⁵ or more.

“Comprising”, “comprises” and “comprised of” are used herein are synonymous with “including”, “includes” or “containing”, “contains”, and are inclusive or open-ended and do not exclude additional, non-recited members, elements or method steps. These terms also encompass “consisting of”.

“Epitope” refers to a specific arrangement of amino acids located on a protein or proteins to which an antibody or antigen-binding fragment thereof or an antibody mimetic bind. Epitopes often consist of a chemically active surface grouping of molecules such as amino acids or sugar side chains, and have specific three-dimensional structural characteristics as well as specific charge characteristics. Epitopes can be linear (or sequential) or conformational, i.e., involving two or more sequences of amino acids in various regions of the antigen that may not necessarily be contiguous.

“Fc domain”, “Fc portion”, and “Fc region” refer to a C-terminal fragment of an antibody heavy chain, e.g., from about amino acid (aa) 230 to about aa 450 of human gamma heavy chain or its counterpart sequence in other types of antibody heavy chains (e.g., α, δ, ε and μ for human antibodies), or a naturally occurring allotype thereof.

“Individual”, or “subject”, refers to an animal, preferably a mammal, more preferably a human. In one embodiment, the individual is a man. In another embodiment, the individual is a woman. In one embodiment, an individual may be a “patient”, i.e., a warm-blooded animal, more preferably a human, who/which is awaiting the receipt of, or is receiving medical care or was/is/will be the object of a medical procedure, or is monitored for the development of cancer. In one embodiment, the individual is an adult (for example a subject above the age of 18). In another embodiment, the individual is a child (for example a subject below the age of 18).

“Polypeptide” refers to any peptide or protein comprising two or more amino acids joined to each other by peptide bonds or modified peptide bonds, i.e., peptide isosteres, thereby forming polymers of amino acids of any length. The polymer may be linear or branched, it may comprise modified amino acids, and it may be interrupted by non-amino acids. The terms also encompass an amino acid polymer that has been modified by, for example, disulfide bond formation, glycosylation, lipidation, acetylation, phosphorylation, pegylation, or any other manipulation, such as conjugation with a labelling component. As used herein the term “amino acid” includes natural and/or unnatural or synthetic amino acids, including glycine and both the D or L optical isomers, and amino acid analogs and peptidomimetics. “Polypeptide” refers to both short chains, commonly referred to as “peptides”, oligopeptides or oligomers, and to longer chains, generally referred to as “proteins”. Hence, within the scope of the present invention, the terms “peptide”, “polypeptide” and “protein” are used interchangeably. Polypeptides may contain amino acids other than the 20 gene-encoded amino acids.

“Preventing cancer” is intended to mean keeping from happening at least one adverse effect or symptom of a cancer.

“Treating cancer” or “treatment” or “alleviation” refers to both therapeutic treatment and prophylactic or preventative measures; wherein the object is to prevent or slow down (lessen) cancer. Those in need of treatment include those already with cancer as well as those prone to have cancer or those in whom cancer is to be prevented. An individual or mammal is successfully “treated” for cancer if, after receiving a therapeutic amount of a polypeptide according to the present invention, the individual shows observable and/or measurable reduction in or absence of one or more of the following: reduction in the number of cancer cells; reduction in the percent of total cells that are cancerous; and/or relief to some extent, one or more of the symptoms associated with cancer; reduced morbidity and mortality, and improvement in quality of life issues. The above parameters for assessing successful treatment and improvement in the cancer are readily measurable by routine procedures familiar to a physician.

“Therapeutically effective amount” is intended to refer to the level or amount of agent that is aimed at, without causing significant negative or adverse side effects to the target, (1) delaying or preventing the onset of cancer; (2) slowing down or stopping the progression, aggravation, or deterioration of one or more symptoms of cancer; (3) bringing about ameliorations of the symptoms of cancer; (4) reducing the severity or incidence of cancer; or (5) preventing cancer formation. In one embodiment, a therapeutically effective amount is administered prior to the onset of cancer formation, for a prophylactic or preventive action. In certain embodiments, the cancer is lung cancer.

“Vaccine” refers to any preparation comprising substance or group of substances meant to cause the immune system of a subject to respond to a given antigen, in particular to a tumor. Prophylactic vaccines are used to prevent a subject from ever having a particular disease, in particular cancer, or to only have a mild case of the disease, in particular cancer. Therapeutic vaccines are intended to treat specific diseases in a subject, in particular cancer. Anti-cancer vaccines comprise a tumor-antigen or tumor-antigens, eliciting an immune response directed against the tumor cells.

“Variant” refers to a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions identical. A variant and reference polypeptide may differ in amino acid sequence by one or more substitutions (preferably conservative), additions, deletions in any combination. A substituted or inserted amino acid residue may or may not be one encoded by the genetic code. A variant of a polynucleotide or polypeptide may be a naturally occurring such as an allelic variant, or it may be a variant that is not known to occur naturally. Non-naturally occurring variants of polynucleotides and polypeptides may be made by mutagenesis techniques or by direct synthesis. Variants should retain one or more of the biological activities of the reference polypeptide.

### DETAILED DESCRIPTION

The present invention relates to a method for treating and/or preventing a cancer in a subject in need thereof, comprising administering to the subject the combination of a therapeutically effective amount of a population of plasmacytoid dendritic cells (PDC) and a therapeutically effective amount of an immune checkpoint inhibitor, wherein said population and said inhibitor may be administered separately or in combination, and in any order.

In some embodiments, the method according to the invention is for vaccination purposes.

In some embodiments, the immune checkpoint inhibitor is an inhibitor of a target selected from the group comprising or consisting of programmed cell death protein-1, ligand of PD-1, cytotoxic T-lymphocyte-associated protein 4 (CTLA-4), T cell immunoglobulin and mucin-domain containing-3 (TIM⁻³), V-domain Ig suppressor of T cell activation (VISTA), lymphocyte-activation gene 3 (LAG-3), T cell immunoglobulin and ITIM domain (TIGIT), B7 homolog 3 protein (B7-H3), B- and T-lymphocyte attenuator (BTLA), Sialic acid binding Ig-like lectin 15 (Siglec-15), cytokine-inducible SH2-containing protein (CISH), and combination thereof.

In some embodiments, the immune checkpoint inhibitor is an inhibitor of programmed cell death protein-1 (PD-1) or an inhibitor of the ligand of PD-1 (PDL-1).

Programmed cell death protein-1 is herein interchangeably referred to as PD-1, PD1, PDCD1, PDCD-1, SLEB2, SLE1 and CD279.

In humans, PD-1 typically has the sequence as disclosed in UniProtKB Ref. Q15116, incorporated herein by reference.

Programmed death-ligand 1 is herein interchangeably referred to as PDL-1, PD-L1, PDL1, PDCDIL1, PDCDILG1, CD274, B7-H1, B7-H, B7H1.

In humans, PD-1 typically has the sequence as disclosed in UniProtKB Ref. Q9NZQ7, incorporated herein by reference.

By “inhibitor”, it is meant that the inhibitor has for biological effect to inhibit or significantly reduce or down-regulate the biological activity of PD-1 or PDL-1. In a particular embodiment, the inhibitor is capable of inhibiting up to about 10%, preferably up to about 25%, preferably up to about 50%, preferably up to about 75%, 80%, 90%, 95%, more preferably up to about 96%, 97%, 98%, 99% or 100% of the activity of PD-1 or PDL-1.

Inhibition of PD-1 or PDL-1 may be assessed by any suitable mean available in the state of the art, in particular any suitable biochemical or biophysical method.

Illustratively, biochemical methods, such as, e.g., affinity electrophoresis, bimolecular fluorescence complementation (BiFC), co-immunoprecipitation, tandem affinity purification, intrinsic tryptophan fluorescence, size exclusion chromatography, fractionated centrifugation, cross-linking (SDS PAGE) electrophoresis; or biophysical methods, such as, e.g., biacore, dual polarization interferometry (DPI), dynamic light scattering (DLS), microscale thermophoresis (MST), NMR WaterLOGSY, Saturation Transfer Difference (STD) spectroscopy, Carr Purcell Meiboom Gill (CPMG) pulse sequence and/or static light scattering (SLS), surface plasmon resonance (SPR) may be employed.

In some embodiments, the inhibitor of PD-1 or PDL-1 is selected from the group comprising or consisting of antibodies and fragments thereof, peptides, nucleic acids, small molecules, pharmacological agents and combinations thereof.

In some embodiments, the inhibitor of PD-1 or PDL-1 is an antibody or a fragment thereof, or a small molecule or a pharmacological agent.

In some embodiments, the inhibitor of PD-1 or PDL-1 is an antibody or a fragment thereof, or a peptide.

In a preferred embodiment the inhibitor of PD-1 or PDL-1 is an antibody or a fragment thereof.

In some embodiments, the inhibitor of the combination for use according to the present invention comprises or consists of a PD-1 inhibitor and/or a PDL-1 inhibitor, preferably the inhibitor of the combination for use according to the present invention comprises or consists of an anti-PD-1 antibody and/or an anti-PDL-1 antibody.

Within the scope of the present invention, it is to be understood that the “antibody” is substantially free of other proteins or antibodies having different antigenic specificities (e.g., an antibody that specifically binds PD-1 or PDL-1 is substantially free of proteins or antibodies that specifically bind antigens other than PD-1 or PDL-1). An antibody that specifically binds PD-1 or PDL-1 may, however, have cross-reactivity to other antigens, such as PD-1 or PDL-1 molecules from other species.

As used herein, a “fragment of an antibody” is preferably intended to refer to an antigen-binding fragment of an antibody.

An antibody or a fragment thereof is said to be “specific for”, “immunospecific” or to “specifically bind” an antigen if it reacts at a detectable level with said antigen (e.g., PD-1 or PDL-1), preferably with an affinity constant (KA) of greater than or equal to about 10⁶ M⁻¹, preferably greater than or equal to about 10⁷ M⁻¹, 10⁸ M⁻¹, 5×10⁸ M⁻¹, 10⁹ M⁻¹, 5×10⁹ M⁻¹ or more. Affinity of an antibody or antigen-binding fragment thereof for its cognate antigen is also commonly expressed as an equilibrium dissociation constant (KD). An antibody or antigen-binding fragment thereof is said to be “immunospecific”, “specific for” or to “specifically bind” an antigen if it reacts at a detectable level with said antigen (e.g., PD-1 or PDL-1), preferably with a K_{D} of less than or equal to 10⁻⁶ M, preferably less than or equal to 10⁻⁷ M, 5×10⁻⁸ M, 10⁻⁸ M, 5×10⁻⁹ M, 10⁻⁹ M or less.

Affinities of antibodies or antigen-binding fragment thereof can be readily determined using conventional techniques. Binding properties of an antibody or antigen-binding fragment thereof to antigens, cells or tissues may generally be determined and assessed using immunodetection methods including, for example, ELISA, immunofluorescence-based assays, such as immuno-histochemistry (IHC) and/or fluorescence-activated cell sorting (FACS) or by surface plasmon resonance (SPR).

In some embodiments, the antibody or fragment thereof is purified.

In some embodiments, the antibody or fragment thereof is purified to greater than 80%, 85%, 90%, 91%, 92%, 93%, 94%, 95% or more by weight of protein or of antibody or antigen-binding fragment thereof, and preferably more than 96%, 97%, 98% or 99% by weight.

In some embodiments, the antibody or fragment thereof is purified to homogeneity (e.g., as shown by SDS-PAGE under reducing or non-reducing conditions and using Coomassie blue or, preferably, silver staining).

In a preferred embodiment, the antibody or fragment thereof is monoclonal. In another embodiment, the antibody or fragment thereof is polyclonal.

In some embodiments, the antibody is whole. In some embodiments, the antibody is truncated. In certain embodiments, the truncated antibody is an antigen-binding fragment; in practice, an antigen-binding fragment retains the ability to bind an epitope on an antigen, but lacks parts of its amino acid sequence that are not involved in epitope binding, e.g., fragment crystallizable region (Fc).

In some embodiments, the antibody or fragment thereof is a recombinant antibody.

In some embodiments, the antibody or fragment thereof, or parts thereof, is comprised in a fusion protein.

In some embodiments, the antibody or fragment thereof is a molecule selected from the group comprising or consisting of a whole antibody, a humanized antibody, a single chain antibody, a dimeric single chain antibody, a Fv, a Fab, a Fab′, a Fab′-SH, a F(ab)′2, a Fd, a defucosylated antibody, a bispecific antibody, unibody, a domain antibody, a nanobody, a diabody, a triabody and a tetrabody.

It will also be appreciated that antibodies or fragments thereof can be modified using known methods. For example, to slow clearance in vivo and obtain a more desirable pharmacokinetic profile, the antibody or antigen-binding fragment thereof may be modified with polyethylene glycol (PEG).

In some embodiments, the antibody or fragment thereof is from the IgG, IgM, IgA, IgD, or IgE class.

In some embodiments, the antibody is a commercially available antibody. In some embodiments, the antibody is approved for clinical trials. In a preferred embodiment, the antibody is authorized for human administration by health authorities, typically the antibody is FDA-approved.

In some embodiments, the inhibitor of PD-1 is an anti-PD-1 antibody or a fragment thereof.

In some embodiments, the inhibitor of PD-1 is selected from the group comprising or consisting of pembrolizumab, nivolumab, lambrolizumab, dostarlimab, cemiplimab, vopratelimab, spartalizumab, camrelizumab, sintilimab, tislelizumab, toripalimab, INCMGA00012, AMP-224, and AMP-514.

In some embodiments, the inhibitor of PD-1 is selected from the group comprising or consisting of pembrolizumab, nivolumab, lambrolizumab, dostarlimab and cemiplimab.

In a preferred embodiment, the inhibitor of PD-1 is pembrolizumab. In another embodiment, the inhibitor of PD-1 is nivolumab. In another embodiment, the inhibitor of PD-1 is lambrolizumab. In another embodiment, the inhibitor of PD-1 is dostarlimab. In another embodiment, the inhibitor of PD-1 is cemiplimab.

In some embodiments, the inhibitor of PD-1 is a combination of at least two anti-PD-1 antibodies.

In some embodiments, the inhibitor of PDL-1 is an anti-PDL-1 antibody or a fragment thereof.

In some embodiments, the inhibitor of PDL-1 is selected from the group comprising or consisting of atezolizumab, durvalumab, avelumab, KN035, Cosibelimab, AUNP12, CA-170, BMS-986189.

In some embodiments, the inhibitor of PDL-1 is selected from the group comprising or consisting of atezolizumab, durvalumab and avelumab.

In one embodiment, the inhibitor of PDL-1 is atezolizumab. In one embodiment, the inhibitor of PDL-1 is durvalumab. In one embodiment, the inhibitor of PDL-1 is avelumab.

In some embodiments, the inhibitor of PDL-1 is a combination of at least two anti-PDL-1 antibodies.

Non-limitative examples of cancer types include carcinoma, lymphoma, blastoma, sarcoma, and leukemia. Within the scope of the present invention, the cancer is preferably, but non-imitatively, a carcinoma, such as e.g., adenocarcinoma, squamous cell carcinoma, squamous cell carcinoma, adenosquamous carcinoma, anaplastic carcinoma, large cell carcinoma, small cell carcinoma, and the like; or a melanoma.

In some embodiments, the cancer is selected from the group comprising or consisting of lung cancer, melanoma, breast cancer, prostate cancer, colon cancer, squamous cell cancer, gastrointestinal cancer, pancreatic cancer, glioblastoma, cervical cancer, ovarian cancer, liver cancer, bladder cancer, colorectal cancer, endometrial carcinoma, salivary gland carcinoma, kidney cancer, vulvar cancer, thyroid cancer, hepatic carcinoma, head and neck cancer, and the like.

In some embodiments, the cancer is lung cancer or melanoma.

In a preferred embodiment, the cancer is lung cancer.

Lung cancer can be distinguished in two main histological categories: small-cell lung cancer (SCLC), that accounts for about 10-20% of all primary lung cancer, and non-small cell lung cancer (NSCLC).

Methods to diagnose lung cancer are known in the art and comprise, without limitation, radiographic screening (X-ray, low-dose helical computed tomography, CT scan), sputum examination, bronchoscopy, and lung tissue biopsy. The diagnosis is preferably performed by a medical practitioner.

In some embodiments, the subject has detectable levels of a lung cancer marker (e.g., at genetic of protein level). In some embodiments, the subject has detectable levels of any of the lung cancer markers selected from the group comprising CYFRA 21-1, carcinoembriogenic antigen (CEA), neuron specific enolase (NSE), and squamous cell carcinoma antigen (SCC-Ag).

In some embodiments, the markers are measured at protein and/or RNA level.

In some embodiments, the markers are measured at protein level. Methods to measure protein expression are known in the art and include, inter alia, enzyme-linked immunosorbent assay (ELISA), Western blot, Dot blot, immunofluorescence, immunochemistry, immunoprecipitation, fluorescent activated cell sorting (FACS), high-performance liquid chromatography (HPLC), and liquid chromatography-mass spectrometry (LC-MS).

In some embodiments, the markers are measured at RNA level. Methods to measure RNA expression are known in the art and include, inter alia, RT-PCR, RT-qPCR, Northern Blot and hybridization techniques.

In some embodiments, the cancer is small-cell lung cancer (SCLC) or non-small cell lung cancer (NSCLC).

In one embodiment, the cancer is NSCLC.

In some embodiments, the NSCLC is selected from the group comprising or consisting of lung adenocarcinoma (LUAD), lung squamous cell carcinoma (LUSC), large-cell carcinoma, and bronchial carcinoid tumor, preferably LUAD.

In another embodiment, the cancer is SCLC.

In another embodiment, the cancer is melanoma.

In some embodiments, the cancer is not melanoma.

In some embodiments, the cancer, preferably lung cancer, is at any stage of the TNM classification, preferably selected among T1a, T1b, T1c, T2a, T2b, T3_{size}, T3ᵢₙᵥ, T3_{centr}, T3ₛₐₜₑₗₗ, T4ᵢₙᵥ, T4ᵢₚₛᵢ, N1, and N2.

In some embodiment, the cancer is at metastasis stage.

In some embodiments, the subject is a smoker or a non-smoker.

Plasmacytoid dendritic cells (PDC) are a subset of dendritic cells. PDC notably are producer of type I interferon and initiators of immune responses, in particular antitumor immune responses.

Methods to isolate, cultivate, and use PDC have been published in Chaperot et al., Journal of immunology vol. 176,1 (2006): 248-55; Lui et al., PloS one vol. 4,9 e7111. 22 Sep. 2009; Aspord et al., The Journal of investigative dermatology vol. 132,10 (2012): 2395-2406; and Hannani et al., International Journal of Molecular Sciences, 2023, 24 (3), 1897, incorporated herein by reference.

In some embodiments, the PDC are cultured in suitable conditions. Methods to culture cells in vitro are well known in the art and are routine practice.

In some embodiments, the PDC are maintained in a controlled atmosphere, i.e., controlled temperature, pressure and composition, typically at 37° C., 1 atm, 5% CO₂.

In some embodiments, the PDC are cultured in a substrate or vessel. In some embodiments, the substrate is plastic. In some embodiments, the plastic substrate is a vessel or container suitable for cell culture, typically a plastic flask or plastic dish.

In one embodiment, the PDC are plated onto a substrate which allows adherence of cells thereto. In certain embodiments, the plastic substrate is treated with at least one agent that promotes cell adherence, survival and/or proliferation; illustratively, in some embodiments, the plastic substrate is treated with poly-D-lysine, gelatin and/or collagen.

In some embodiments, the agent forms a layer or a matrix at the surface of the plastic substrate.

In another embodiment, the PDC are maintained in a substrate which prevents adherence of cells thereto. In some embodiments, the PDC are cultured in suspension.

In some embodiments, the PDC are cultured in a culture medium sustaining their further proliferation, generally a liquid culture medium, which may contain serum or may be serum-free.

In some embodiments, the PDC are growth in bioreactor in suspension in synthetic medium.

Culture media are known in the art. The term “culture medium” or “cell culture medium” or “medium” refers to an aqueous liquid or gelatinous substance comprising nutrients which can be used for maintenance or growth of cells. Cell culture media can contain serum or be serum-free. These media, that can be further supplemented with appropriate mixture of organic or inorganic compounds may, besides providing nutrients and/or growth promoters, also promote the e growth/adherence or the elimination/detachment of specific cell types.

In some embodiments, the culture medium comprises a basal medium formulation as known in the art and include, without limitation, Eagle's Minimum Essential Medium (MEM), OPTI-MEM, Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, Medium 199, Waymouth's MB 752/1 X-VIVO-15, or Williams Medium E, and modifications and/or combinations thereof. Compositions of the above basal media are generally known in the art and it is within the skill of one in the art to modify or modulate concentrations of media and/or media supplements as necessary for culturing the PDC.

In a preferred embodiment, the culture medium used to cultivate the population of PDC is X-VIVO-15, or variants thereof.

By means of illustration and not limitation, these ingredients may include inorganic salts (in particular salts containing Na, K, Mg, Ca, Cl, P and possibly Cu, Fe, Se and Zn), physiological buffers (e.g., HEPES, bicarbonate), nucleotides, nucleosides and/or nucleic acid bases, ribose, deoxyribose, amino acids, vitamins, antioxidants (e.g., glutathione) and sources of carbon (e.g., glucose, pyruvate, e.g., sodium pyruvate, acetate, e.g., sodium acetate), etc. It will also be apparent that many media are available as low-glucose formulations with or without sodium pyruvate.

In some embodiments, basal media are supplemented with one or more further components selected from the group comprising or consisting of transferrin, selenium salts, amino acids, sugar, and combinations thereof. These components can be included in a salt solution such as, but not limited to, Hanks' Balanced Salt Solution (HBSS), Earle's Salt Solution. Further antioxidant supplements may be added, e.g., β-mercaptoethanol. While many basal media already contain amino acids, some amino acids may be supplemented later, e.g., L-glutamine, which is known to be less stable when in solution.

In some embodiments, the culture medium is further supplemented with antibiotic and/or antimycotic compounds, such as, typically, mixtures of penicillin and streptomycin, and/or other compounds, exemplified but not limited to, amphotericin, ampicillin, gentamycin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, zeocin, and combinations thereof.

In some embodiments, the culture medium is further supplemented with fungicide compounds.

In some embodiments, the culture medium is further supplemented with hormones selected from the group comprising or consisting of D-aldosterone, diethylstilbestrol (DES), dexamethasone, estradiol, hydrocortisone, insulin, prolactin, progesterone, somatostatin/human growth hormone (HGH), thyrotropin, thyroxine, L-thyronine, epithelial growth factor (EGF), human recombinant epidermal growth factor, and combinations thereof. The final concentration of hormones may range from 0.01 to 1000 ng/mL, preferably from 0.1 to 100 ng/mL, more preferably from 1 to 10 ng/mL.

In some embodiments, the culture medium is further supplemented with lipids and lipid carriers selected from the group comprising or consisting of cyclodextrin, cholesterol, linoleic acid conjugated to albumin, linoleic acid and oleic acid conjugated to albumin, unconjugated linoleic acid, linoleic-oleic-arachidonic acid conjugated to albumin, oleic acid unconjugated and conjugated to albumin, and the like, and combinations thereof. Albumin can similarly be used in fatty-acid free formulations.

In some embodiments, the culture medium is further supplemented with mammalian plasma or serum. In some embodiments, the concentration of the plasma or serum in the culture medium is from 0.01% to 20%, preferably from 0.1% to 10%, more preferably from 1% to 5%. In some embodiments, the concentration of the plasma or serum in the culture medium is about 1%, about 2%, about 3%, about 4%, about 5%, about 6%, about 7%, about 8%, about 9%, or about 10%.

The term “serum”, as conventionally defined, is obtained from a sample of whole blood by first allowing clotting to take place in the sample and subsequently separating the so formed clot and cellular components of the blood sample from the liquid component (serum) by an appropriate technique, typically by centrifugation. An inert catalyst, e.g., glass beads or powder, can facilitate clotting. Advantageously, serum can be prepared using serum-separating vessels (SST), which contain the inert catalyst to mammals. Plasma or serum often contains cellular factors and components that are necessary for viability and expansion.

In some embodiments, the plasma or serum for use in the culture medium as described herein may include human plasma or serum; or plasma or serum derived from non-human animals, preferably non-human mammals, such as, e.g., non-human primates (e.g., lemurs, monkeys, apes), fetal or adult bovine, horse, porcine, lamb, goat, dog, rabbit, mouse or rat serum or plasma, etc. In some embodiments, the culture medium is further supplemented with a serum replacement or analogue.

As appreciated by those skilled in the art, the cultured PDC may be counted in order to facilitate re-seeding of the PDC at a desired density.

In some embodiments, the PDC are cultured by re-seeding them at a density of between 100 and 10⁵ cells/cm², and at a splitting ratio between about 1/100 and 1/2. As used herein, the splitting ratio denotes the fraction of the passaged PDC that is seeded into an empty (typically a new) culture vessel of the same surface area and/or volume as the vessel from which the PDC were obtained.

The type of culture vessel, as well as of surface and/or volume allowing PDC proliferation into the culture vessel and the cell culture media, can be the same as initially used and as described hereinabove, or may be different.

In some embodiments, the population of PDC is devoid of contamination by bacteria, fungi, protozoa, archaea, or other microbial species.

In some embodiments, the PDC are stored for future use.

In some embodiments, PDC are cryopreserved for storage. In some embodiments, PDC are stored at a temperature from −20° C. to −200° C., preferably from −80° C. to −200° C. In some embodiments, the PDC are stored at about −196° C., typically cells are stored in liquid nitrogen. In some embodiments, PDC are “flash-frozen” in liquid nitrogen prior to storage.

In some embodiments, the PDC are contacted with at least one cryoprotective agent prior to cryopreservation. Cryoprotective agent are known in the art and include, e.g., glycerol, sucrose, serum and the like.

In some embodiments, the population of PDC is characterized in that it is substantially pure.

In some embodiments, the substantially pure population of PDC comprises less than 50%, 40%, 30%, 20%, 10%, 5%, 4%, 3%, 2%, 1%, 0.5%, 0.1%, 0.01%, 0.001% or less of other contaminating cell type(s).

In some embodiments, the substantially pure population of PDC comprises 0% of other contaminating cell type(s). In some embodiments, the substantially pure cell population comprises no more than one cell type.

In a preferred embodiment, the population of PDC is of clinical grade.

In some embodiments, the population of PDC is irradiated prior to human administration. In some embodiments, the dose of irradiation is between 10 and 100 Gy. In a preferred embodiment, the dose of irradiation is between 30 and 60 Gy.

In some embodiments, the PDC express major histocompatibility complex (MHC) antigen specific to humans.

In some embodiments, the PDC express MHC of class I such as human leukocyte antigen (HLA). In some embodiments, HLA comprises or consists of HLA-A, HLA-B, and/or HLA-C subtypes.

In some embodiments, HLA is a heterodimer and is composed of a heavy a chain and smaller β chain. In some embodiments, the a chain is encoded by a variant HLA-A gene, generating HLA-A serotype group. In some embodiments, HLA serotypes are selected from the list comprising or consisting of HLA-A*02:01, HLA-A*02:02, HLA-A*02:03, HLA-A*02:05, HLA-A*02:06, HLA-A*02:11.

In a certain embodiment, the PDC express HLA-A. In a preferred embodiment, PDC express HLA-A2 subtype. In a more preferred embodiment, PDC express HLA-A*02:01 serotype.

In some embodiments, the PDC are genetically modified.

As used herein, “genetically modified” means that the genome of the PDC comprises at least one modification consisting of a mutation and/or an insertion of one or more nucleic acid sequences. Methods to genetically modify a cell are known in the art and include, non-imitatively, CRIPR-Cas-based techniques, Zinc finger nucleases-based techniques, TALEN and the like. In a preferred embodiment, the PDC are constitutively modified, i.e., the modification of the genome is transmitted to daughter cells, typically the PDC are transduced by methods known in the art such as lentiviral vectors. In another, less preferred embodiment, the PDC are transiently modified, typically the PDC are transfected.

In some embodiments, the PDC are genetically modified to express at least one protein of interest.

As used herein, “at least one” means 1, 2, 3, 4, 5, 6, 7, 8, 9, 10, 11, 12, 13, 14, 15, 6, 17, 18, 19 or 20.

As used herein, “genetically modified to express at least one protein of interest” means that the PDC expresses 1.2-fold, 1.5-fold, 2-fold, 3-fold, 5-fold, 10-fold or more the at least one protein of interest, or transcript encoding thereof, compared to an unmodified PDC. In some embodiments, the PDC overexpress the at least one protein of interest.

In some embodiments, the at least one protein of interest is a secreted protein or a surface protein.

In some embodiments, the at least one protein of interest is a secreted protein. In some embodiments, the secreted protein is a cytokine. In some embodiments, the cytokine is an interleukin. In some embodiments, the interleukin is selected from the group comprising or consisting of IL-2, IL-7, IL-12 and IL-15. In some embodiments, the interleukin is IL-12 or IL-15.

In some embodiments, the at least one protein of interest is IL-12.

In humans, IL-12 typically comprises 2 subunits, namely IL12A (or p35) and IL12B (or p40), typically having the sequence of Uniprot Ref. P29459 and Uniprot Ref. P29460, respectively, incorporated herein by reference. IL-12 is interchangeably referred to as IL-12, IL12, interleukin 12, and p70.

In some embodiments, the at least one protein of interest is IL-15.

In humans, IL-15 typically has the sequence as disclosed in Uniprot Ref. P40933, incorporated herein by reference. IL-15 is interchangeably referred to as IL-15, IL15, and interleukin 15.

In some embodiments, the at least one protein of interest is IL-7.

In humans, IL-7 typically has the sequence as disclosed in Uniprot Ref. P13232, incorporated herein by reference. IL-7 is interchangeably referred to as IL-7, IL7, and interleukin 7.

In some embodiments, the at least one protein of interest is a surface protein.

In some embodiments, the surface protein is of the B7 family of proteins. In some embodiments, the surface protein from the B7 family of proteins is CD80 or CD86.

In some embodiments, the at least one protein of interest is CD80.

In humans, CD80 typically has the sequence as disclosed in Uniprot Ref. P33681, incorporated herein by reference. CD80 is interchangeably referred to as CD80, B7, B7-1, B7.1, BB1, CD28LG, CD28LG1, and LAB7.

In some embodiments, the at least one protein of interest is CD86.

In humans, CD86 typically has the sequence as disclosed in Uniprot Ref. P42081, incorporated herein by reference. CD86 is interchangeably referred to as CD86, B7-2, B7.2, B70, CD28LG2, and LAB72.

In some embodiments, the at least one protein of interest is mutated. In some embodiments, the at least one protein of interest comprises at least one amino acid mutation compared to the wild type protein of interest.

As used herein, “at least one” means 1, 2, 3, 4, 5, 6, 7, 8, 9, 10, 11, 12, 13, 14, 15, 6, 17, 18, 19 or 20. In some embodiments, “amino acid mutation” comprises substitutions, deletions, insertions, inversions, and combination thereof.

In some embodiments, the mutations of the at least one protein of interest do not produce any adverse effect on the PDC, i.e., the mutations of the at least one protein of interest do not alter the viability, and/or proliferation capacities of the PDC. In some embodiments, the mutations of the at least one protein of interest are not oncogenic.

In some embodiments, the mutations of the at least one protein of interest increase at least 1.5-fold, 2-fold, 3-fold or more the efficiency of the PDC as antigen presenting cells. In some embodiments, the mutations of the at least one protein of interest increase at least 1.5-fold, 2-fold, 3-fold or more the viability and/or circulating time of the PDC.

In some embodiments, the PDC are genetically modified to reduce (knock-down) or abolish (knock-out) the expression of at least one protein or gene encoding thereof.

As used herein, “reduce” means a decrease of expression from 10%, 20%, 30%, 40%, 50%, 60%, 70%, 80%, 90%, 95%, and at most 99%, of the basal expression of the at least one protein or gene encoding thereof. As used herein, “reduce” means a 100% decrease of expression of the basal expression of the at least one protein or gene encoding thereof.

As antigen presenting cells, PDC are capable of activating naïve T cells and memory T cells into activated, antigen-specific T cells, either by direct priming, or by cross priming (i.e., presentation of exogenously-derived antigens). This second mechanism allows PDC to be contacted, loaded, pulsed, charged or the like, with any given antigen of interest or combination of antigens of interest. The loaded PDC activate T cells specific for the antigen of interest or combination of antigens of interest.

In some embodiments, the population of PDC is contacted with at least one cancer antigen and/or fragment and/or variant thereof.

As used herein, the term “contacted” is intended to mean that the population of PDC is incubated with one or several cancer antigens and/or fragment and/or variant thereof, resulting in the “loading” of the one or several cancer antigens and/or fragment and/or variant thereof on the population of PDC. Within the scope of the present invention, the terms “contacted”, “loaded”, or “incubated” may be used interchangeably. This incubation itself is hereby interchangeably referred to as the “loading step”.

In some embodiments, the incubation is performed in a culture medium as described herein, or in a biological buffer, preferably in a culture medium, more preferably in X-VIVO-15 culture medium.

In some embodiments, the incubation is performed at a temperature from 32° C. to 42° C., preferably at 37° C.

In some embodiments, the incubation is performed for a duration from 30 minutes to 24 hours.

In some embodiments, the incubation is performed for a duration from 1 hour to 24 hours, from 2 hours to 24 hours, from 4 hours to 24 hours, from 8 hours to 24 hours, or from 16 hours to 24 hours.

In some embodiments, the incubation is performed for a duration from 30 minutes to 16 hours, from 30 minutes to 8 hours, from 30 minutes to 4 hours, or from 30 minutes to 2 hours.

In some embodiments, the incubation is performed for a duration from 1 hour to 16 hours, from 2 hours to 8 hours, or from 2 hours to 4 hours.

In a preferred embodiment, the incubation is performed for about 3 hours.

In some embodiments, the cellular concentration of the population of PDC used for the loading step is 1×10², 1×10³, 1×10⁴, 1×10⁵, 1×10⁶, 1×10⁷, 1×10⁸, 1×10⁹, or 1×10¹⁰ cells/mL. In some embodiments, the PDC cellular concentration is comprised between 1×10⁴ and 1×10⁸ cells/mL. In a preferred embodiment, the PDC cellular concentration is about 1×10⁶ cells/mL.

In some embodiments, the concentration of the cancer antigen and/or fragment and/or variant thereof used for the loading step is comprised between 1 μM and 10 μM final. In some embodiments, the concentration of the cancer antigen and/or fragment and/or variant thereof used for the loading step is comprised between 2 μM and 5 μM final

In some embodiments, β2-microglobulin is also added to the loading step.

In some embodiments, the concentration of B2-microglobulin used for the loading step is comprised between 0.1 and 1 μg/mL. In a preferred embodiment, the concentration of B2-microglobulin is about 0.1 μg/mL.

In some embodiments, the population of PDC is irradiated after the loading step. In some embodiments, the dose of irradiation is between 10 and 100 Gy. In a preferred embodiment, the dose of irradiation is between with 30 and 60 Gy.

In some embodiments, the population of PDC is substantially devoid of free cancer antigen and/or fragment and/or variant thereof, i.e., about 100% of the cancer antigen and/or fragment and/or variant thereof is loaded on the population of PDC.

In some embodiments, the at least one cancer antigen and/or fragment and/or variant thereof is not expressed in healthy lung tissues. In some embodiments, the at least one cancer antigen and/or fragment and/or variant thereof have low expression levels in healthy tissues, preferably healthy lung tissues.

In some embodiments, the at least one cancer antigen and/or fragment and/or variant thereof is a molecule selected from the group comprising or consisting of peptides, polypeptides, proteins, polysaccharides, lipids, nucleic acids, and combinations thereof.

In some embodiments, the at least one cancer antigen and/or fragment and/or variant thereof is a molecule selected from the group comprising or consisting of peptides, polypeptides, and proteins.

In a preferred embodiment, the at least one cancer antigen and/or fragment and/or variant thereof is a peptide or polypeptide comprising at least 3, at least 5, at least 10, at least 15, at least 20, or more, amino acid residues.

In some embodiments, the peptide comprises natural and non-natural amino acids. In a preferred embodiment, the peptide comprises or consists of natural amino acids. In another embodiment, the peptide comprises or consists of non-natural amino acids.

In some embodiments, the peptide comprises at least one post-translational modification on at least one amino acid residues. In some embodiments, the peptide is glycosylated, glycated, phosphorylated, biotinylated, oxidated, nitrated, nitrosylated, acylated, alkylated, acetylated, methylated, lipidated, ubiquitinated, carboxylated, pegylated, or combinations thereof.

In another embodiment, the at least one cancer antigen and/or fragment and/or variant thereof is a polysaccharide. In another embodiment, the at least one cancer antigen and/or fragment and/or variant thereof is a lipid. In another embodiment, the at least one cancer antigen and/or fragment and/or variant thereof is a nucleic acid molecule.

In some embodiments, the at least one cancer antigen and/or fragment and/or variant thereof elicits an immune response, i.e., the at least one cancer antigen and/or fragment and/or variant thereof is immunogenic. In a preferred embodiment, the at least one cancer antigen and/or fragment and/or variant thereof elicits an adaptative immune response. In a more preferred embodiment, the at least one cancer antigen and/or fragment and/or variant thereof elicits T cell-mediated immune response.

In some embodiments, the at least one cancer antigen and/or fragment and/or variant thereof is selected from the group comprising or consisting of MAGE-A1, MAGE-A2, MAGE-A3, MAGE-A4, MAGE-A9, MAGE-A10, CAMEL (CTAG2), GLULD1 (LGSN), HER2 (ERBB2), MUC1, SURVIVIN (BIRC5), NY-ESO-1, MULTI-MAGE, NY-BR-1, Cyclin D1 (CCND1), PD-L1, CEA, EPCAM, IDO, LY-6K, MUC5AC, and 5T4 (TPBG).

In a preferred embodiment, the at least one cancer antigen and/or fragment and/or variant thereof is selected from the group comprising or consisting of MAGE-A1, MAGE-A3, MAGE-A4, MAGE-A9, MUC1, SURVIVIN (BIRC5), NY-ESO-1, CAMEL, MULTI-MAGE, 5T4 (TPBG) and CCND1.

In another embodiment, the at least one cancer antigen and/or fragment and/or variant thereof is selected from the group comprising or consisting of NY-ESO-1, CAMEL, MAGE-A2, MAGE-A3, and MAGE-A9.

In some embodiments, the at least one cancer antigen and/or fragment and/or variant thereof is MAGE-A1. In some embodiments, the at least one cancer antigen and/or fragment and/or variant thereof is MAGE-A3. In some embodiments, the at least one cancer antigen and/or fragment and/or variant thereof is MAGE-A4. In some embodiments, the at least one cancer antigen and/or fragment and/or variant thereof is MAGE-A9. In some embodiments, the at least one cancer antigen and/or fragment and/or variant thereof is MUC1. In some embodiments, the at least one cancer antigen and/or fragment and/or variant thereof is SURVIVIN (BIRC5). In some embodiments, the at least one cancer antigen and/or fragment and/or variant thereof is NY-ESO-1. In some embodiments, the at least one cancer antigen and/or fragment and/or variant thereof is CAMEL (CTAG2). In some embodiments, the at least one cancer antigen and/or fragment and/or variant thereof is MULTI-MAGE. In some embodiments, the at least one cancer antigen and/or fragment and/or variant thereof is 5T4 (TPBG). In some embodiments, the at least one cancer antigen and/or fragment and/or variant thereof is CCND1.

Without wanting to be bound to a theory, the inventors expect the population of PDC and/or the immune checkpoint inhibitors to exert at least one biological effect on the organism of the subject and/or on each other.

In some embodiments, the population of PDC stimulates peripheral blood mononuclear cells, more preferably said population of PDC stimulates peripheral blood mononuclear cells with tumor-specific/antitumor activity.

In some embodiments, the population of PDC stimulates or activates circulating CD8+ T cells.

It is also known in the field that stimulation or activation of circulating CD8+ T cells by dendritic cells (DC) in vivo results from the “priming” process. “Priming” is defined as the ability of DC to stimulate naive T cells, i.e., that have never seen an antigen, with a higher stimulation or activation threshold than memory cells. In one embodiment, the T cells, in particular CD8+ T cells, are antigen-primed by the PDC of the invention. As a result of such a specific and durable contact T cell-to-PDC contact, a naïve T cell may become activated and subsequently proliferate and differentiate into effector forms.

In some embodiments, the CD8+ T cells are activated in Antigen Specific CD8+ T-Cells (ASTC). It will be apparent to the person skilled in the art that the “antigen” corresponds to the at least one cancer antigen and/or fragment and/or variant thereof, as described herein.

In some embodiments, the at least one anti-PD-1 antibody amplifies at least 1.5-fold the expansion of activated CD8+ T cells, preferably said at least one anti-PD-1 antibody amplifies at least 1.5-fold the expansion of ASTC.

Within the scope of the invention, the at least 1.5-fold means at least 1.5, 2.0, 2.5, 3.0, 3.5, 4.0, 4.5, 5.0, 5.5, 6.0, 6.5, 7.0, 7.5, 8.0, 8.5, 9.0, 9.5, 10, 15, 20, 25, 30, 35, 40, 45, 50, 55, 60, 65, 70, 75, 80, 85, 90, 95, 100, 150, 200, 300, 400, 500, 1000-fold, or more.

In a preferred embodiment, the at least one anti-PD-1 antibody amplifies at least 2-fold the expansion of activated CD8+ T cells, preferably said at least one anti-PD-1 antibody amplifies at least 2-fold the expansion of ASTC.

In some embodiments, the method according to the invention increases at least 1.5-fold, 2-fold, 3-fold or more oncolytic activity of immune cells in the subject.

The method according of the invention comprises administering a therapeutically effective dose of the population of PDC and/or the immune checkpoint inhibitor according to the invention. The meaning of the expression “therapeutically effective dose” within the scope of the present invention is detailed hereinafter.

In one embodiment, the population of PDC is administered at a dose from 50,000 cells/antigen and/or fragment and/or variant thereof to 15 million cells/antigen and/or fragment and/or variant thereof. In another embodiment, the population of PDC is administered at a dose from 50,000 cells/antigen and/or fragment and/or variant thereof to 5 million cells/antigen and/or fragment and/or variant thereof.

In certain embodiments, the population of PDC is administered at a dose from 50,000 cells/antigen and/or fragment and/or variant thereof to 4 million, 3 million, 2 million, 1 million, 900,000, 800,000, 700,000, 600,000, 500,000, 400,000, 300,000, 200,000, or 100,000 cells/antigen and/or fragment and/or variant thereof.

In certain embodiments, the population of PDC is administered at a dose from 100,000, 150,000, 200,000, 250,000, 300,000, 350,000, 400,000, 450,000 cells/antigen and/or fragment and/or variant thereof to 5 million cells/antigen and/or fragment and/or variant thereof.

In a preferred embodiment, the population of PDC is administered at a dose from 500,000 cells/antigen and/or fragment and/or variant thereof to 5 million cells/antigen and/or fragment and/or variant thereof.

In certain embodiments, the population of PDC is administered at a dose from 750,000 cells/antigen and/or fragment and/or variant thereof to 4 million cells/antigen and/or fragment and/or variant thereof, from 1 million cells/antigen and/or fragment and/or variant thereof to 3 million cells/antigen and/or fragment and/or variant thereof.

In a preferred embodiment, the population of PDC is administered at a dose of about 2 million cells/antigen and/or fragment and/or variant thereof.

In another embodiment, the population of PDC is administered at a dose from 1,000 to 100,000 cells per kg of body weight.

In some embodiments, the population of PDC is administered at a dose from 5,000 to 100,000 cells per kg of body weight, from 10,000 to 100,000 cells per kg of body weight, from 50,000 to 100,000 cells per kg of body weight.

In some embodiments, the population of PDC is administered at a dose from 1,000 to 50,000 cells per kg of body weight, from 1,000 to 10,000 cells per kg of body weight, from 1,000 to 5,000 cells per kg of body weight.

In some embodiments, the population of PDC is administered at a dose from 2,000 to 50,000 cells per kg of body weight, from 5,000 to 40,000 cells per kg of body weight, from 10,000 to 30,000 cells per kg of body weight, from 15,000 to 25,000 cells per kg of body weight. In some embodiments, the population of PDC is administered at a dose of about 20,000 cells per kg of body weight.

Methods for cell counting are known in the art and comprise, without limitation, hemocytometer, automated cell counter, flow cytometer, spectrophotometry and the like.

In some embodiments, the immune checkpoint inhibitor is administered at a dose of at least 0.01 mg/kg, preferably at least 0.1 mg/kg, more preferably at least 1 mg/kg, even more preferably at least 3 mg/kg.

In some embodiments, the immune checkpoint inhibitor is administered at a dose of at most 1 g/kg, preferably at most 100 mg/kg, more preferably at most 10 mg/kg.

In some embodiments, the immune checkpoint inhibitor is administered at a dose from 0.1 mg/kg to 100 mg/kg, preferably from 0.25 mg/kg to 80 mg/kg, more preferably from 0.50 mg/kg to 60 mg/kg, even more preferably from 0.75 mg/kg to 40 mg/kg, even more preferably from 1 mg/kg to 20 mg/kg, even more preferably from 2 mg/kg to 15 mg/kg.

In a preferred embodiment, the immune checkpoint inhibitor is administered at a dose from 3 to 10 mg/kg.

In some embodiments, the immune checkpoint inhibitor is administered at a dose of 3 mg/kg. In some embodiments, the immune checkpoint inhibitor is administered at a dose of 4 mg/kg. In some embodiments, the immune checkpoint inhibitor is administered at a dose of 5 mg/kg. In some embodiments, the immune checkpoint inhibitor is administered at a dose of 6 mg/kg. In some embodiments, the immune checkpoint inhibitor is administered at a dose of 7 mg/kg. In some embodiments, the immune checkpoint inhibitor is administered at a dose of 8 mg/kg. In some embodiments, the immune checkpoint inhibitor is administered at a dose of 9 mg/kg. In some embodiments, the immune checkpoint inhibitor is administered at a dose of 10 mg/kg.

In one embodiment, the population of PDC is administered at least one time.

In a preferred embodiment, the population of PDC is administered at least 3 times.

In a more preferred embodiment, the population of PDC is administered at least 6 times.

Within the scope of the present invention, the expression “at least one time” is intended to mean at least 1 time, at least 2 times, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, or more. Within the scope of the present invention, the expression “at least 3 times” is intended to mean at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, or more. Within the scope of the present invention, the expression “at least 6 times” is intended to mean at least 6 times, at least 7 times, at least 8 times, at least 9 times, at least 10 times, at least 11 times, at least 12 times, at least 13 times, at least 14 times, at least 15 times, or more.

In some embodiments, the population of PDC is administered from 1 time to 10 times.

In some embodiments, the population of PDC is administered from 2 times to 10 times, from 3 times to 10 times, from 4 times to 10 times, from 5 times to 10 times, from 6 times to 10 times, from 7 times to 10 times, from 8 times to 10 times, or from 9 times to 10 times.

In some embodiments, the population of PDC is administered from 2 times to 9 times, from 3 times to 8 times, from 4 times to 7 times, or from 5 times to 6 times.

In some embodiments, the population of PDC is administered 1 time, 2 times, 3 times, 4 times, 5 times, 6 times, 7 times, 8 times, 9 times, 10 times, or more. In one embodiment, the population of PDC is administered 3 times. In a preferred embodiment, the population of PDC is administered 6 times.

In some embodiments, the immune checkpoint inhibitor is administered at least one time.

In a preferred embodiment, the immune checkpoint inhibitor is administered at least 3 times.

In a more preferred embodiment, the immune checkpoint inhibitor is administered at least 6 times.

Within the scope of the present invention, the expression “at least one time” is intended to mean at least 1 time, at least 2 times, at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, or more. Within the scope of the present invention, the expression “at least 3 times” is intended to mean at least 3 times, at least 4 times, at least 5 times, at least 6 times, at least 7 times, at least 8 times, at least 9 times, at least 10 times, at least 11 times, at least 12 times, at least 13 times, at least 14 times, at least 15 times, or more. Within the scope of the present invention, the expression “at least 6 times” is intended to mean at least 6 times, at least 7 times, at least 8 times, at least 9 times, at least 10 times, at least 11 times, at least 12 times, at least 13 times, at least 14 times, at least 15 times, or more.

In some embodiments, the immune checkpoint inhibitor is administered from 1 time to 100 times. In some embodiments, the immune checkpoint inhibitor is administered from 2 times to 100 times, from 3 times to 100 times, from 4 times to 100 times, from 5 times to 100 times, from 6 times to 100 times, from 7 times to 100 times, from 8 times to 100 times, or from 9 times to 100 times.

In some embodiments, the immune checkpoint inhibitor is administered up to 90 times, up to 80 times, up to 70 times, up to 60 times, up to 50 times, up to 40 times, up to 30 times, up to 20 times.

In some embodiments, the immune checkpoint inhibitor is administered 1 time, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, or more. In one embodiment, the immune checkpoint inhibitor is administered 3 times. In a preferred embodiment, the immune checkpoint inhibitor is administered 6 times.

In a preferred embodiment, the immune checkpoint inhibitor is administered until progression. By “progression”, it is meant alleviation of at least one symptom of the cancer. In some embodiments, progression corresponds to a 1.5-fold, 2-fold, 5-fold, 10-fold decrease of expression, or more, of at least one cancer marker. In some embodiments, progression corresponds to a 1.5-fold, 2-fold, 5-fold, 10-fold increase in Quality-of-Life Scale (QOLS), or more. Progression of the treatment and/or evolution of the disease is to be assessed by health practitioners.

In another embodiment, the immune checkpoint inhibitor is administered until clinical benefit. As used herein, the term “clinical benefit” means a favorable effect on a meaningful aspect of how the subject feels (e.g., symptom relief), functions (e.g., reduction of cancer cells) or survives as a result of treatment. Clinical benefit may be measured as an improvement or delay in the progression of a disease, preferably a cancer.

In some embodiments, clinical benefit corresponds to an increase in activated CD8+ T cells, preferably ASTC, as described herein. Clinical benefit in the disease is to be assessed by health practitioners.

In some embodiments, at least one blood sampling is performed in the subject. It is to be understood that the blood sample is for use for further analysis and for follow up of the treatment, in particular for immunomonitoring.

In some embodiments, the at least one blood sample is centrifuged at 300 to 1000×g for 10 to 30 minutes. In some embodiments, the at least one blood sample is mixed with Ficoll-Paque medium prior to centrifugation.

In certain embodiments, centrifugation results in the formation of more than one fraction (e.g., red blood cells, platelets, plasma, peripheral blood mononuclear cells etc.). In some embodiments, the peripheral blood mononuclear cells (PBMC) fraction is substantially isolated. In some embodiments, the PBMC fraction is substantially purified.

In some embodiments, the PBMC fraction is frozen. In some embodiments, the PBMC fraction is frozen at −20° C., −80° C. or −196° C., preferably −196° C. In some embodiments, the PBMC fraction is frozen in liquid nitrogen. In a preferred embodiment, PBMC fraction is contacted with at least one suitable cryoprotectant agent known in the art.

In some embodiments, the frozen PBMC are thawed prior to analysis.

In some embodiments, the at least one blood sample, PBMC, thawed PBMC and/or purified CD8+ T cells are analyzed, assessed, quantified, or screened for CD8+ T cells, preferably activated CD8+ T cells, and more preferably ASTC.

In some embodiments, CD8+ T cells, preferably CD8+CD3+ T cells, are purified from the PBMC or the thawed PBMC, by any suitable means known in the art. In a preferred embodiment CD8+ T cells, preferably CD8+CD3+ T cells, are purified from the PBMC or the thawed PBMC by magnetic-activated cell sorting (MACS).

In some embodiments, CD8+ T cells, preferably activated CD8+ T cells, and more preferably ASTC, are further submitted to multimer staining. In some embodiments, multimer staining is performed by contacting CD8+ T cells, preferably activated CD8+ T cells, and more preferably ASTC with at least one multimer or dextramer comprising one or more HLA-A2/peptide complexes, wherein the at least one multimer or dextramer is specific for the at least one cancer antigen and/or fragment and/or variant thereof.

In some embodiments, CD8+ T cells, preferably activated CD8+ T cells, and more preferably ASTC, are incubated with fluorochrome-conjugated antigen-specific dextramers. In some embodiments, the proportion of the CD8+ T cells, preferably activated CD8+ T cells, and more preferably ASTC is assessed by flow cytometry. In some embodiments, CD8+ T cells, preferably activated CD8+ T cells, and more preferably ASTC, are sorted by any suitable means known in the art, preferably by fluorescence-activated cell sorting (FACS).

In some embodiments, a viability or living cell marker is used in flow cytometry and/or FACS analysis, known in the art. In a preferred embodiment, the viability or living marker is a fixable viability stain 510 Dye.

In some embodiments, the at least one blood sample, PBMC, thawed PBMC and/or purified CD8+ cells are analyzed, assessed, quantified, or screened for differentiation markers. In some embodiments, the differentiation markers are CD45RA and/or CCR7.

In some embodiments, the at least one blood sample, PBMC, thawed PBMC and/or purified CD8+ cells are analyzed, assessed, quantified, or screened for activation markers. In some embodiments, the activation markers are CD25, HLA-DR and/or CD54.

In some embodiments, the at least one blood sample is analyzed, assessed, quantified, or screened for cancer markers.

In some embodiments, the method according to the present invention further comprises a step of testing the subject for cancer markers, preferably testing at least one blood sample for cancer markers. In some embodiments, the method according to the present invention further comprises a step of numbering activated CD8+ T cells, preferably ASTC, in the subject.

In some embodiments, the method according to the present invention further comprises a step of assessing and/or quantifying the efficacy of the treatment. In some embodiments, the method according to the present invention further comprises a step of concluding if the treatment should be maintained, continued, adapted, halted, suspended, stopped, modified or resumed, based on the assessment and/or quantification of the efficacy of the treatment.

In some embodiments, the population of PDC is administered every week, every 2 weeks, every 3 weeks, every 4 weeks, every 5 weeks, every 6 weeks, every 7 weeks, every 8 weeks, every 9 weeks, or every 10 weeks.

In a preferred embodiment, the population of PDC is administered every week, every 2 weeks, or every 3 weeks.

In a more preferred embodiment, the population of PDC is administered every week.

In some embodiments, the immune checkpoint inhibitor is administered every week, every 2 weeks, every 3 weeks, every 4 weeks, every 5 weeks, every 6 weeks, every 7 weeks, every 8 weeks, every 9 weeks, or every 10 weeks.

In a preferred embodiment, the immune checkpoint inhibitor is administered every 2 weeks, every 3 weeks, or every 4 weeks.

In a more preferred embodiment, the immune checkpoint inhibitor is administered every 3 weeks.

Within the scope of the present invention, a period of one week is meant to be understood as expiring in the subsequent week on the day having the same name as the day on which the administration was performed.

In some embodiments, the population of PDC and the immune checkpoint inhibitor may be administered separately or in combination, and in any order.

In one embodiment, the population of PDC and the immune checkpoint inhibitor are administered in combination.

In some embodiments, the population of PDC and the immune checkpoint inhibitor are both administered within 24 hours, 18 hours, 12 hours, 6 hours, 5 hours, 4 hours, 3 hours, 2 hours, 1 hour, 30 minutes, 15 minutes, 10 minutes or 5 minutes. In some embodiments, the administration of the population of PDC and the administration of the immune checkpoint inhibitor are separated by at most 24 hours, 18 hours, 12 hours, 6 hours, 5 hours, 4 hours, 3 hours, 2 hours, 1 hour, 30 minutes, 15 minutes, 10 minutes or 5 minutes.

In some embodiments, the administration of the population of PDC and the administration of the immune checkpoint inhibitor are performed sequentially, i.e., one after the other. In certain embodiments, less than 5 minutes separate the administration of the population of PDC and the administration of the immune checkpoint inhibitor.

In some embodiments, the population of PDC and the immune checkpoint inhibitor are administered concomitantly. In one embodiment, the population of PDC and the immune checkpoint inhibitor are administered by 2 simultaneous administrations, typically 2 simultaneous injections. In another embodiment, the population of PDC and the immune checkpoint inhibitor are administered in a single administration, typically a single injection.

In another embodiment, the population of PDC and the immune checkpoint inhibitor are administered separately.

In some embodiments, the population of PDC is administered 1 day, 2 days, 3 days, 4 days, 5 days, 6 days, 1 week, 2 weeks, 3 weeks, 4 weeks or more after the immune checkpoint inhibitor. In some embodiments, the immune checkpoint inhibitor is administered 1 day, 2 days, 3 days, 4 days, 5 days, 6 days, 1 week, 2 weeks, 3 weeks, 4 weeks or more after the population of PDC.

For the purposes of the method for treating and/or preventing a cancer in a subject in need thereof according to the present invention, the administration of the population of PDC and the immune checkpoint inhibitor may follow a specific course of events.

Thus, in another embodiment, the administration of the population of PDC and the administration of the immune checkpoint inhibitor is performed according to a specific sequence, order, or protocol of administration.

In some embodiments, the sequence, order, or protocol of administration starts with the administration of a therapeutically effective dose of the population of PDC or a therapeutically effective dose of the immune checkpoint inhibitor.

It is to be understood that, within the scope of the present invention, the week of the first administration is “the first week”. Subsequent and consecutive weeks are hereby referred to as “1 week after the start”, “2 weeks after the start”, “3 weeks after the start” and so on, or alternatively and interchangeably, as “the second week”, “the third week”, “the fourth week” and so on. A group of weeks consisting of “the first week” and any number (n) of subsequent and consecutive weeks is hereby referred to as “the first n weeks”.

In a preferred embodiment, the sequence, order, or protocol of administration starts with the concomitant administration of a therapeutically effective dose of the population of PDC and a therapeutically effective dose of the immune checkpoint inhibitor.

In some embodiments, a therapeutically effective dose of the population of PDC is administered every week, every 2 weeks, every 3 weeks or more after the start of the sequence, order, or protocol of administration. In a preferred embodiment, a therapeutically effective dose of the population of PDC is administered every week after the start of the sequence, order, or protocol of administration.

In some embodiments, a therapeutically effective dose of the population of PDC is administered for up to 9 weeks, 8 weeks, 7 weeks, 6 weeks, 5 weeks, 4 weeks, 3 weeks, 2 weeks, or 1 week after the start of the sequence, order, or protocol of administration.

In a preferred embodiment, a therapeutically effective dose of the population of PDC is administered up to 6 weeks, 5 weeks, 4 weeks, or 3 weeks after the start of the sequence, order, or protocol of administration. In a more preferred embodiment, a therapeutically effective dose of the population of PDC is administered up to 5 weeks after the start of the sequence, order, or protocol of administration, i.e., the total number of administrations of the population of PDC is 6.

In some embodiments, a therapeutically effective dose of the immune checkpoint inhibitor is administered every week, every 2 weeks, every 3 weeks, every 4 weeks, every 5 weeks or more after the start of the sequence, order, or protocol of administration. In a preferred embodiment, a therapeutically effective dose of the immune checkpoint inhibitor is administered every 3 weeks after the start of the sequence, order, or protocol of administration.

In a preferred embodiment, a therapeutically effective dose of the immune checkpoint inhibitor is administered until progression, as defined herein. In some embodiments, the immune checkpoint inhibitor is administered for up to 100 weeks, 90 weeks, 80 weeks, 70 weeks, 60 weeks, 50 weeks, 40 weeks, 30 weeks, 20 weeks, or 10 weeks after the start of the sequence, order, or protocol of administration.

In some embodiments, the sequence, order, or protocol of administration is characterized in that a therapeutically effective dose of the population of PDC is administered for the first week, first 2 weeks, first 3 weeks, first 4 weeks, first 5 weeks, first 6 weeks, preferably a therapeutically effective dose of the population of PDC is administered for the first 3 weeks, more preferably the population of PDC is administered for the first 6 weeks.

In some embodiments, the sequence, order, or protocol of administration is characterized in that:
a therapeutically effective dose of the population of PDC is administered for the first week, first 2 weeks, first 3 weeks, first 4 weeks, first 5 weeks, first 6 weeks, preferably the population of PDC is administered for the first 3 weeks, more preferably the population of PDC is administered for the first 6 weeks, and
a therapeutically effective dose of the population of PDC is administered every week.

In some embodiments, the sequence, order, or protocol of administration is characterized in that a therapeutically effective dose of the immune checkpoint inhibitor is administered at least during the first week. In some embodiments, the sequence, order, or protocol of administration is characterized in that a therapeutically effective dose of the immune checkpoint inhibitor is administered at least during the first week, and the third week. In some embodiments, the sequence, order, or protocol of administration is characterized in that a therapeutically effective dose of the immune checkpoint inhibitor is administered at least during the first week, the third week and the sixth week. In some embodiments, the sequence, order, or protocol of administration is characterized in that a therapeutically effective dose of the immune checkpoint inhibitor is administered at least during the first week, the third, week the sixth week and the ninth week.

In some embodiments, the sequence, order, or protocol of administration is characterized in that:
a therapeutically effective dose of the population of PDC is administered for the first week, first 2 weeks, first 3 weeks, first 4 weeks, first 5 weeks, first 6 weeks, preferably the population of PDC is administered for the first 3 weeks, more preferably the population of PDC is administered for the first 6 weeks,
a therapeutically effective dose of the population of PDC is administered every week, and
a therapeutically effective dose of the immune checkpoint inhibitor is administered at least during the first week and until progression.

In some embodiments, the sequence, order, or protocol of administration is characterized in that:
a therapeutically effective dose of the population of PDC is administered every week for the first 6 weeks, and
a therapeutically effective dose of the immune checkpoint inhibitor is administered every 3 weeks until progression.

In some embodiments, the sequence, order, or protocol of administration is preceded by the collection of a blood sample of the subject. In some embodiments, the sequence, order, or protocol of administration further comprises the collection of a blood sample of the subject, at any time point after the first administration of the population of PDC and/or the first administration of the immune checkpoint inhibitor.

In some embodiments, the immune checkpoint inhibitor and/or said population of PDC are administered intravenously, subcutaneously, intradermally, intraarterially, intraperitoneally, intramuscularly and/or intratumorally.

In a preferred embodiment, the immune checkpoint inhibitor and/or the population of PDC are administered intravenously and/or subcutaneously. In a preferred embodiment, the immune checkpoint inhibitor and/or the population of PDC are administered intravenously and subcutaneously.

In one embodiment, the immune checkpoint inhibitor is administered intravenously. In another embodiment, the immune checkpoint inhibitor is administered subcutaneously.

In another embodiment, the immune checkpoint inhibitor is administered both intravenously and subcutaneously. In certain embodiments, the dose administered intravenously is identical to the dose administered subcutaneously, i.e., the dose administered by each route is equal to half of the total dose administered to the subject. In certain embodiments, the dose administered intravenously is different from the dose administered subcutaneously, wherein the sum of the doses administered by each route is equal to the total dose administered to the subject.

In one embodiment, the population of PDC is administered intravenously. In another embodiment, the population of PDC is administered subcutaneously.

In another embodiment, the population of PDC is administered both intravenously and subcutaneously. In certain embodiments, the dose administered intravenously is identical to the dose administered subcutaneously, i.e., the dose administered by each route is equal to half of the total dose administered to the subject. In certain embodiments, the dose administered intravenously is different from the dose administered subcutaneously, wherein the sum of the doses administered by each route is equal to the total dose administered to the subject.

In one embodiment, the immune checkpoint inhibitor and the population of PDC are administered by the same route.

In another embodiment, the immune checkpoint inhibitor and the population of PDC are administered by distinct routes. In certain embodiments, the dose administered by a first route (typically, intravenously) is the same as the dose administered by a second route (typically, subcutaneously). In another embodiment, the dose administered by a first route is higher or lower than the dose administered by a second route.

In some embodiments, the immune checkpoint inhibitor and/or said population of PDC are comprised in a solution suitable for human administration, in particular human systemic injection (e.g., saline solution with 0.9% sodium chloride).

In some embodiments, the method further comprises administering to said subject at least one additional anti-cancer agent.

In some embodiments, the at least one additional anti-cancer agent is a pharmacologically active molecule, e.g., a small molecule, or a biologically active peptide/protein. In some embodiments, the at least one additional anti-cancer agent is comprised in pharmacological composition approved (e.g., FDA or EMA approved) for the treatment of at least one disease. In some embodiments, the at least one additional anti-cancer agent is undergoing at least one preclinical or clinical trial.

In a preferred embodiment, the at least one additional anti-cancer agent targets at least one gene selected from the list comprising or consisting of EGFR, ALK, ROS1, BRAF, RET, MET, and HER2.

Anticancer agents are known from the state of the art. Non-limitative examples of anticancer agents include acalabrutinib, alectinib, alemtuzumab, anastrozole, avapritinib, avelumab, belinostat, bevacizumab, bleomycin, blinatumomab, bosutinib, brigatinib, carboplatin, carmustine, cetuximab, chlorambucil, cisplatin copanlisib, cytarabine, daunorubicin, decitabine, dexamethasone, docetaxel, doxorubicin, encorafenib, erdafitinib, etoposide, everolimus, exemestane, fludarabine, 5-fluorouracil, gemcitabine, ifosfamide, imatinib Mesylate, leuprolide, lomustine, mechlorethamine, melphalan, methotrexate, mitomycin, nelarabine, paclitaxel, pamidronate, panobinostat, pralatrexate, prednisolone, ofatumumab, rituximab, temozolomide, topotecan, tositumomab, trastuzumab, vandetanib, vincristine, vorinostat, zanubrutinib, and the likes.

In certain embodiments, the at least one anti-cancer agent is to be administered in combination with, concomitantly or sequentially, the combination for use according to the invention.

In some embodiments, the method further comprises administering to said subject at least one adjuvant.

Adjuvants are known in the art and comprise, non-initiatively, amorphous metals (e.g., aluminum), altered metals (e.g., aluminum hydroxide, aluminum phosphate, aluminum potassium sulfate, aluminum hydroxyphosphate sulfate and the like), lipids (e.g., oils; monophosphoryl lipid A and the like), nucleic acids (e.g., cytosine phosphoguanine), saponins or other chemicals.

It will be appreciated that adjuvants are preferentially used preventively, i.e., in order to elicit an immune response against an antigen or fragment or variant thereof, prior to the onset of a disease, in particular cancer.

In some embodiments, the method further comprises a step of assessing the immune background of the subject by blood sampling.

In some embodiments, the step of step of assessing the immune background of the subject consists of numbering activated CD8+ T cells, preferably ASTC, in the subject, and/or measuring their activity, proliferation and/or cytotoxic potential, preferably oncolytic potential.

In some embodiments, the method further comprises a step of assessing the immunogenicity of the subject by blood sampling at least one time post-vaccination. Blood sampling and analysis have been described herein.

The present invention further relates to a composition comprising or consisting of a population of PDC as described herein, and an immune checkpoint inhibitor as described herein.

The present invention further relates to a combination of a population of PDC as described herein, with an immune checkpoint inhibitor as described herein.

The present invention further relates to a combination of a population of PDC, and at least one immune checkpoint inhibitor, for use for the treatment and/or prevention of cancer in a subject in need thereof.

The present invention further relates to a population of plasmacytoid dendritic cells (PDC) for use for treating and/or preventing cancer, wherein the population of PDC is for administration or is administered in combination with at least one immune checkpoint inhibitor.

The present invention further relates to a pharmaceutical composition comprising a population of plasmacytoid dendritic cells (PDC) and at least one immune checkpoint inhibitor for treating and/or preventing cancer in a subject in need thereof.

In some embodiments, the pharmaceutical composition further comprises at least one pharmaceutically acceptable vehicle or excipient.

In some embodiments, the pharmaceutically acceptable vehicle or excipient is selected in a group comprising or consisting of a solvent, a diluent, a carrier, an excipient, a dispersion medium, a coating, an absorption delaying agent and any combinations thereof. The carrier, diluent, solvent or excipient must be “acceptable” in the sense of being compatible with the population of PDC and/or the immune checkpoint inhibitor, and not be deleterious upon being administered to a subject. Typically, the vehicle or excipient does not produce an adverse, allergic or other untoward reaction when administered to a subject.

In some embodiments, the population of PDC and the at least one immune checkpoint inhibitor are associated with distinct pharmaceutically acceptable vehicles or excipients.

The present invention further relates to a kit comprising a population of plasmacytoid dendritic cells (PDC) and at least one immune checkpoint inhibitor for treating and/or preventing cancer in a subject in need thereof.

The present invention further relates to the use of a population of PDC as described herein, and an immune checkpoint inhibitor as described herein, for the manufacture of a medicament for treating and/or preventing cancer.

In some embodiments, the medicament further comprises at least one pharmaceutically acceptable vehicle or excipient as described herein.

### BRIEF DESCRIPTION OF THE DRAWINGS

FIG. 1 is a schematic view of the design of the study.

FIG. 2 is a set of dot plots showing the gating strategy for the study. Lymphocytes were selected based on their morphology in SSC versus FSC dot plots. Then, singlets living cells were successively selected and outliner dots were excluded on the CD45RA versus CCR7 dot plots. Then early and late events were excluded on time dot plot. Finally, CD3+CD8+ T-cells were selected on CD3 versus CD8 dot pot before gating the population of multimer-positive cells to determine the frequency of tumor antigen-specific CD8+ T cells. As three different multimer tools are used for three peptide-specific CD8+ T-cells, the determination of the positivity of one antigen-specific CD8+ T cell population is determined by the mean of the proportion of cells gated in tow displays. The example shows the proportion of EBV-specific CD8+ T cells with 4.32% of positive cells gated in dextramer 1 versus dextramer 2 dot plot, and also 4.32% of positive cells gated in dextramer 1 versus dextramer 3 dot plot.

FIGS. 3A-3B are a combination of graphs showing patients' response to PDC*line cells in combination or not with anti-PD1. Two cohorts of patients were studied: cohort A1, treated with PDC*line cells, and cohort B1, treated with PDC*line cells and anti-PD1 antibodies. Blood sampling was performed before and after treatment. The proportion of ASTC (among CD8+ T cells) was measured by flow cytometry. Antigens NY-ESO-I (NY), MAGE-A3 (M3), MULTIMAGE (MM), SURVIVIN(S) and MELAN-A (MEL-A) were studied. FIG. 3A is an illustrative dot plot showing the proportion of ASTC for 3 positive patients of cohort A1 (P6, P7 and P9). FIG. 3B is an illustrative dot plot showing the proportion of ASTC for 4 positive patients of cohort B1 (P1, P4, P5 and P7). Each dot represents an expansion of ASTC. If several expansions were seen for the same patient, all expansions were represented. Non positive patients are not shown.

FIGS. 4A-4B are a combination of graphs showing patients' response to PDC*line cells in combination or not with anti-PD1 (see FIG. 2). Blood sampling were performed after 1 weeks (V7), 4 weeks (V8) and 10 weeks (V9) from the last dose of PDC*line cells. The proportion of ASTC (CD8+ T cells) was measured by flow cytometry. FIG. 4A is an illustrative dot plot showing the fold change of ASTC compared to baseline for 3 positive patients of cohort A1 (P6, P7 and P9). FIG. 4B is an illustrative dot plot showing the fold change of ASTC compared to baseline for 4 positive patients of cohort B1 (P1, P4, P5 and P7). Each dot represents an expansion of ASTC. If several expansions were seen for the same patient, all expansions were represented. Non positive patients are not shown.

FIG. 5 is a graph showing the fold change of the percentage of Antigen-Specific T cells (ASTC) after 1 weeks (V7), 4 weeks (V8) and 10 weeks (V9) from the last dose of PDC*line cells compared to baseline before treatment.

### EXAMPLES

The present invention is further illustrated by the following examples.

### Example 1: Synergized Effect of Anti-PD1 with PDC*Line to Expand Antitumor CD8+ T Cells

### Materials and Methods

### Preparation of Tumor-Peptide-Loaded PDC*Line

PDC*line cells were separately loaded with a distinct synthetic peptide encoded by a lung tumor antigen, namely NY-ESO-1 (NY), MAGE-A3 (M3), Multi-MAGE (MM, an epitope common to MAGE-A1, -A2, -A3, -A4, -A6, -A10, and -A12 antigens), SURVIVIN(S) or a peptide derived from the Melan-A antigen (MelA) used as immunogenic positive control. Briefly, PDC*line cells (1 million/mL) were incubated for 3 h at 37° C. with 10 μM of each peptide. The tumor peptides were purchased from PolyPeptide Laboratories, Inc. The characteristics of the peptides are detailed in Table 1. After loading, the cells were washed, irradiated and stored frozen as cryovials until use.

TABLE 1
List of the selected tumor antigens.

| | Peptide Sequence | SEQ |
|---|---|---|
| Antigen | (HLA-A*02:01) | ID NO |
| MAGE-A3 | FLWGPRALV | 1 |
| MULTI-MAGE | YLEYRQVPV | 2 |
| NY-ESO-1 (CTAG1B) | SLLMWITQC | 3 |
| SURVIVIN (BIRC5) | LMLGEFLKL | 4 |
| MELAN-A | ELAGIGILTV | 5 |

### Patients Treatment and Peripheral Blood Samplings

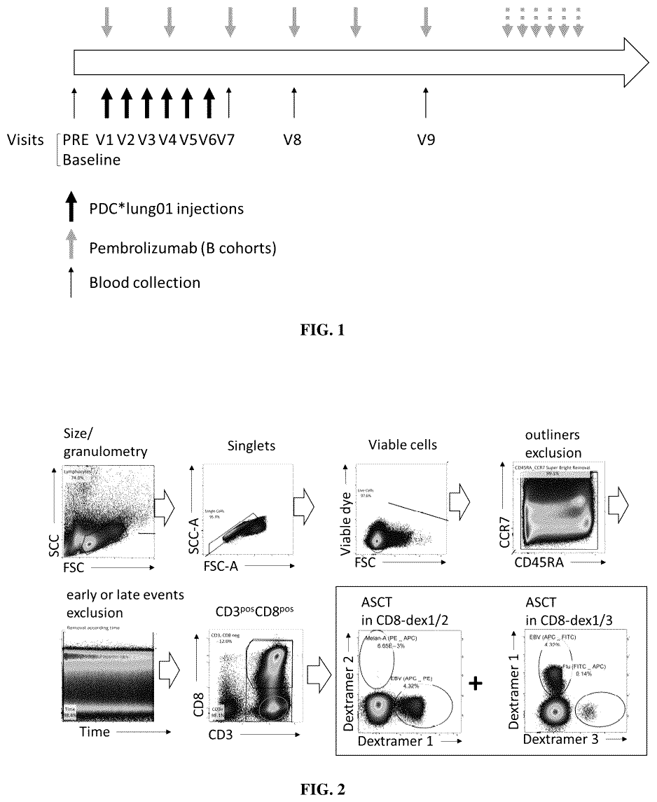

PDC*line cells were administered as single agent (cohort A1) or added to an anti-PD-1 (cohort B1). Patients of cohort A1 were treated on a weekly basis at each of the six treatment visits, with 2 million of PDC*line cells per loaded tumor antigen (i.e., 2 million of PDC*line cells loaded with NY, 2 million loaded with M3, etc. . . . ), 1 million administered subcutaneously and 1 million administered intravenously. Patients of cohort B1 were also treated on a weekly basis at each of the six treatment visits, with 2 million of PDC*line cells per loaded tumor antigen (1 million administered subcutaneously and 1 million administered intravenously), but in addition to 200 mg Pembrolizumab. The first PDC*line injection started within 48 hours after the first infusion of anti-PD-1. Pembrolizumab has been administered every 3 weeks (FIG. 1).

Patients are different from cohort A1 and from cohort B1. For each cohort, patients were numbered P1, P2, P3 . . . .

A pre-blood sampling analysis was performed before the experiment in order to assess the basal percentage of antigen specific T cell (ASTC) in each patient. Peripheral blood samplings were collected after 1 week (V7), 4 weeks (V8) and 10 weeks (V9) from the last PDC*line administration. PBMCs were purified from the patients' blood using Ficoll-Hypaque density gradient centrifugation (Lymphocyte Separation Medium, Eurobio) and stored frozen until analysis.

### Detection of Antigen-Specific CD8+ T Cells

The percentage of ASTC expansion was assessed by flow cytometry after each blood sampling. After thawing the samples, PBMCs were subjected to a red blood cells lysis. Samples were then subjected to a CD8+ T cells purification using MACS technology. Briefly, the magnetic labelling was performed as followed: PBMC pellet was resuspended in 40 μl of Purification Buffer per 10×10⁶ total PBMC and incubated with 10 μl of Biotin-Antibody Cocktail per 10×10⁶ total PBMC during 5 minutes. Then, 30 μl of Purification Buffer and 20 μl of CD8+ T Cell Microbead Cocktail per 10×10⁶ total PBMC were added and incubated for 15 minutes at 4° C. A CD8+ T Cells Magnetic Separation was then performed as followed: PBMCs suspension was applied onto each LS column and flow-through containing unlabeled cells (representing enriched CD8+ T cells) was collected. Column was then washed 3 times with Purification Buffer in order maximized the collection of CD8+ T cells. Magnetically labelled cells (non CD8+ T cells) were flushed out of the column with Purification Buffer.

Enriched samples in terms of CD8+ T Cells were then subjected to dextramer labelling. Briefly, cells were centrifuged then cell pellet was resuspended in PBS-2% FBS−2 mM EDTA+10 μM Biotin at a concentration of 1×10⁶ viable cell/ml. Cells were then incubated with a fixable viability stain 510 Dye, 0.5 μl for 1.00×10⁶ of viable cells and incubated for 20 minutes. After washing steps, cells were incubated with fluorochrome-conjugated antigen-specific dextramers (PE Dextramer MAGE-A3 Cat: WB3415-PE; PE Dextramer Survivin Cat: WB2167-PE; FITC Dextramer NY-ESO-1 Cat: WB3247-FITC; APC Dextramer Multi-MAGE Cat: WB3880-APC; PE Dextramer Melan-A Cat: WB2162-PE-Immudex, Denmark) for 20 min in the dark at room temperature. After washing, BUV737 Mouse Anti-Human CD3 Antibody (BD Biosciences, Cat: 612752), BV421 Mouse Anti-Human CD8 Antibody (BD Biosciences, Cat: 562428), BUV395 Mouse Anti-Human CD45RA Antibody (BD Biosciences, Cat: 740315) and BV785 Mouse Anti-Human CCR7 Antibody (BioLegend, Cat: 353230) were added and incubated for 20 min in the dark at 4° C. Cells were then washed and resuspended in FACS Lysing solution before fluorescence acquisition with a flow cytometer (BD FACS Aria III). The frequency of multimer-positive cells was measured in the CD8-positive living single-cell population of lymphocytes.

Patients' PBMCs and the cells collected at the end of cocultures were resuspended in PBS (Gibco, Life Technologies, France) with 2% decomplemented FCS and incubated with fluorochrome-conjugated antigen-specific multimers for 20 min in the dark at room temperature. Either tetramers (ITag, Beckman Coulter, Villepinte) or dextramers (Immudex, Denmark) were used as multimers.

After washing, BV421-conjugated anti-CD3 (BD Biosciences, Le Pont de Claix, France) and PerCP-Cy5.5-conjugated anti-CD8 antibodies (BD Biosciences, Le Pont de Claix, France) were added and incubated for 20 min in the dark at 4° C. Viability dye (Live and Dead, Fisher Scientific, Illkirch, France) was added to the antibody mix. The cells were washed and resuspended in FACS Lysing solution (BD Biosciences, #349202) before fluorescence acquisition with a flow cytometer (BD FACS Canto II) and analysis with FlowJo software (Tree Star, Inc., Ashland, OR, USA). The frequency of multimer-positive cells was measured in the CD8-positive living single-cell population of lymphocytes, as described in FIG. 2.

The limit of quantification of ASTCs was set at 0.005% of total gated CD8+ T cells for MAGE-A3-, Survivin-, and Melan-A-specific CD8+ T-cells and 0.003% for the other specificities.

### Results

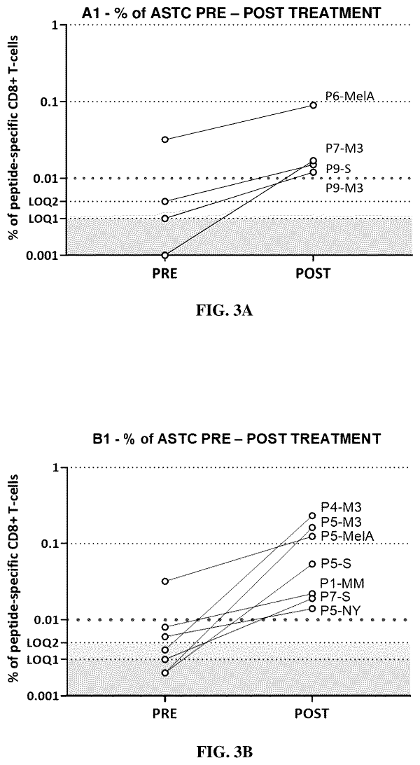

The inventors showed that the combination of PDC*line cells with anti-PD1 leads to higher number of patients showing an immune response and a higher magnitude of this response than PDC*lung01 alone (FIGS. 3A, 3B).

Results revealed an increase in the percentage of antigen-CD8 T-cells (ASTC) following treatment with the combination of PDC*line with anti-PD1.

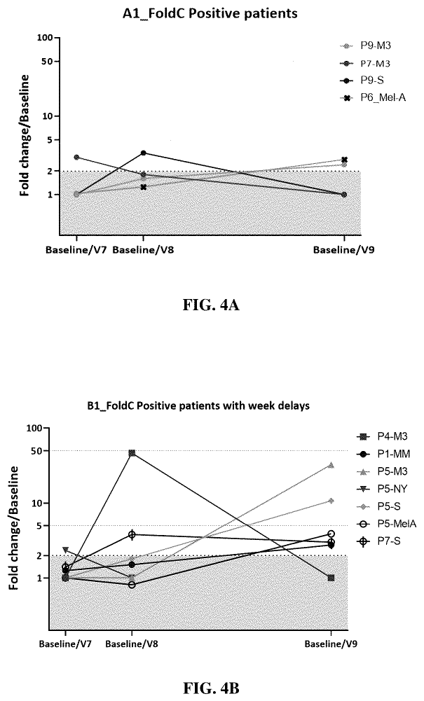

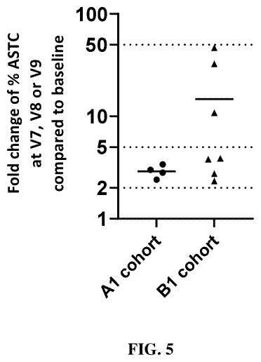

Results from cohort A1 showed that 3/6 patients treated had an increase of ASTC (50% positive patients) and 2 lung antigens responses were detected (M3, S) (FIG. 4A and corresponding Table 2) while results from cohort B1 showed 4/6 patients treated had an increase of ASTC (67% positive patients) and 3 lung antigens responses were detected (MM, M3, S) (FIG. 4B and corresponding Table 3).

TABLE 2
Cohort Al positive patients' response to lung antigen after treatment.

| Visits | A1-Patients | | | | | | TOTAL |
|---|---|---|---|---|---|---|---|
| Patients | P03 | P04 | P05 | P06 | P07 | P09 | 6 |
| V7 | — | — | — | — | M3 | — | 1 |
| V8 | — | — | — | — | | S | 1 |
| V9 | — | — | — | MelA | 1 | M3 | 2 |
| Total | 0 | 0 | 0 | 1 | 1 | 2 | |

TABLE 3
Cohort B1 positive patients' response
to lung antigen after treatment.

| Visits | B1-Patients | | | | | | TOTAL |
|---|---|---|---|---|---|---|---|
| Patients | P01 | P02 | P03 | P04 | P05 | P07 | 6 |
| V7 | — | — | — | — | — | — | 0 |
| V8 | — | — | — | M3 | — | S | 2 |
| V9 | — | — | — | — | MelA, M3, | S | 5 |
| | | | | | NY, S | | |
| Total | 1 | 0 | 0 | 1 | 4 | 1 | |

Results without MelA data revealed that only 2/6 patients (33% positive patients) are responding to PDC*line antigen alone for cohort A1, while the ratio of positive patients remains the same for the cohort B1, demonstrating the efficacity of PDC*line antigen combined with anti-PD1. These results show that the combination of PDC*line with anti-PD1 has a synergic effect to expand antitumor CD8+ T Cells.

Results also revealed that PDC*line with anti-PD1 induced an immune response against all the peptides.

TABLE 4
Comparison of the lung antigens immune
response in cohort A1 and B1.

| | Number | | | | | |
|---|---|---|---|---|---|---|
| | of | Lung Antigens | | | | |
| | analyzed | MAGE- | | NY- | Multi- | |
| Cohort | patients | A3 | Survivin | ESO-I | MAGE | Melan-A |
| A1 | 6 | 2 | 1 | 0 | 0 | 1 |
| B1 | 6 | 2 | 2 | 1 | 1 | 1 |

1-24. (canceled)

25. A method for treating and/or preventing a cancer in a subject in need thereof, comprising administering to the subject the combination of a therapeutically effective amount of a population of plasmacytoid dendritic cells (PDC) and a therapeutically effective amount of an immune checkpoint inhibitor, wherein said population and said inhibitor may be administered separately or in combination, and in any order.

26. The method according to claim 25, wherein said immune checkpoint inhibitor is an inhibitor of programmed cell death protein-1 (PD-1) or an inhibitor of the ligand of PD-1 (PDL-1).

27. The method according to claim 26, wherein said inhibitor of PD-1 is an anti-PD-1 antibody or a fragment thereof.

28. The method according to claim 26, wherein said inhibitor of PD-1 is selected from the group comprising or consisting of pembrolizumab, nivolumab, lambrolizumab, dostarlimab and cemiplimab.

29. The method according to claim 26, wherein said inhibitor of PDL-1 is an anti-PDL-1 antibody or a fragment thereof.

30. The method according to claim 26, wherein said inhibitor of PDL-1 is selected from the group comprising or consisting of atezolizumab, durvalumab and avelumab.

31. The method according to claim 25, wherein said cancer is selected from the group comprising or consisting of lung cancer, melanoma, breast cancer, prostate cancer, colon cancer, squamous cell cancer, gastrointestinal cancer, pancreatic cancer, glioblastoma, cervical cancer, ovarian cancer, liver cancer, bladder cancer, hepatoma, colorectal cancer, endometrial carcinoma, salivary gland carcinoma, kidney cancer, vulvar cancer, thyroid cancer, hepatic carcinoma, head and neck cancer, and the like.

32. The method according to claim 25, wherein said cancer is lung cancer or melanoma.

33. The method according to claim 25, wherein said cancer is small-cell lung cancer (SCLC) or non-small cell lung cancer (NSCLC).

34. The method according to claim 25, wherein cancer is NSCLC.

35. The method according to claim 25, wherein said population of PDC is contacted with at least one cancer antigen and/or fragment and/or variant thereof.

36. The method according to claim 35, wherein said at least one cancer antigen and/or fragment and/or variant thereof is selected from the group consisting of MAGE-A1, MAGE-A2, MAGE-A3, MAGE-A4, MAGE-A9, MAGE-A10, CAMEL (CTAG2), GLULD1 (LGSN), HER2 (ERBB2), MUC1, SURVIVIN (BIRC5), NY-ESO-1, MULTI-MAGE, NY-BR-1, Cyclin D1 (CCND1), PD-L1, CEA, EPCAM, IDO, LY-6K, MUC5AC, and 5T4 (TPBG).

37. The method according to claim 25, wherein said population of PDC is administered at least one time.

38. The method according to claim 25, wherein said population of PDC is administered from 1 time to 10 times.

39. The method according to claim 25, wherein said immune checkpoint inhibitor is administered at least one time.

40. The method according to claim 25, wherein said immune checkpoint inhibitor and/or said population of PDC are administered intravenously, subcutaneously, intraarterially, intradermally, intratumorally, intraperitoneally and/or intramuscularly.

41. The method according to claim 25, further comprising administering to said subject at least one additional anti-cancer agent.

42. The method according to claim 25, further comprising administering to said subject at least one adjuvant.

43. The method according to claim 25, further comprising a step of assessing the immune background of said subject by blood sampling.

44. The method according to claim 25, further comprising a step of assessing the immunogenicity of the subject by blood sampling at least one time post-vaccination.


SEQUENCE LISTING (5 sequences)

SEQ ID NO: 1 - protein, 9 aa, synthetic construct
FLWGPRALV

SEQ ID NO: 2 - protein, 9 aa, synthetic construct
YLEYRQVPV

SEQ ID NO: 3 - protein, 9 aa, synthetic construct
SLLMWITQC

SEQ ID NO: 4 - protein, 9 aa, synthetic construct
LMLGEFLKL

SEQ ID NO: 5 - protein, 10 aa, synthetic construct
ELAGIGILTV